(19) United States
(12) Patent Application Publication
(10) Pub. No.: US 2026/0309622 A1
(43) Pub. Date: Oct. 8, 2026

(54) REAL-TIME INLINE MILK ANALYSIS SYSTEM AND METHOD

(71) Applicants: MICHAEL MCHUGH, PLYMOUTH, MN (US); CHRISTOPHER MICHAEL ANDERSON, MINNEAPOLIS, MN (US)

(72) Inventors: MICHAEL MCHUGH, PLYMOUTH, MN (US); CHRISTOPHER MICHAEL ANDERSON, MINNEAPOLIS, MN (US)

(21) Appl. No.: 19/460,194

(22) Filed: Jan. 26, 2026

Related U.S. Application Data
(63) Continuation-in-part of application No. 17/985,862, filed on Nov. 13, 2022, now abandoned.

(51) Int. Cl. G01N 21/359; G01N 33/06

(52) U.S. Cl. CPC G01N 21/359; G01N 33/06

(57) ABSTRACT

A system and method for determining a DHIA-equivalent milk analyte concentration from inline near-infrared (NIR) measurements taken during a single milking session of a dairy animal, including receiving a time-series of validated NIR spectrograms acquired at least every five seconds. Included is applying a regression model to generate a time-series of predicted analyte concentrations c(tᵢ); automatically detecting and trimming pre-milk and post-milk portions; mathematically transforming the trimmed time-series into a single DHIA-equivalent value using a transformation function trained to replicate constant-volume drip sampling of a DHIA jar; and outputting the single DHIA-equivalent value.

### CLAIM OF PRIORITY

This continuation-in-part application claims priority to and the benefit of U.S. patent application Ser. No. 17/985,862, filed on Nov. 13, 2022, titled DAIRY HERD IMPROVEMENT TESTING METHOD AND SYSTEM, which claims priority to U.S. Provisional Application 63/279,170, filed on Nov. 14, 2021, also titled DAIRY HERD IMPROVEMENT TESTING METHOD AND SYSTEM, which are herein incorporated by reference in their entirety.

### FIELD OF THE INVENTION

The present disclosure generally relates to a real-time, inline milk analysis system for determining a DHIA-equivalent milk analyte concentration.

### BACKGROUND

The dairy industry is a critical component of global agriculture, with milk being a valuable commodity subject to stringent regulations on quality, cleanliness, and quantity. Traditional milk quality testing relies on the physical collection of proportional milk samples into a Dairy Herd Improvement Association (DHIA) jar during an entire milking. The jar mechanically mixes constant-volume drips taken throughout the milking session, yielding one homogeneous sample per cow per test day (typically monthly). That sample is preserved and shipped to a certified laboratory for such tests as wet-chemistry analysis. While accurate, this process is expensive (approximately $25-$35 per sample in 2025), slow (2-7-day turnaround), and provides only a single aggregated data point per cow per month.

Therefore, there is a long-felt need in the market for an improved on-site in-line milk analyzer.

### SUMMARY OF THE INVENTION

Disclosed is a method for determining a DHIA-equivalent milk analyte concentration from inline near-infrared (NIR) measurements taken during a single milking session of a dairy animal, comprising: receiving a time-series of validated NIR spectrograms acquired at least every five seconds. Included are tests such as applying a regression model to generate a time-series of predicted analyte concentrations c(tᵢ); automatically detecting and trimming pre-milk and post-milk portions; mathematically transforming the trimmed time-series into a single DHIA-equivalent value using a transformation function trained to replicate constant-volume drip sampling of a DHIA jar; and outputting the single DHIA-equivalent value.

The transformation function may be a clipped rectangular integration over the central 70-95% of the milking interval. The transformation function may be a weighted average Σ wᵢ c(tᵢ)/Σ wᵢ, the weights wᵢ empirically optimized to minimize deviation from physical DHIA jar results. The analyte may be selected from fat, protein, lactose, progesterone, β-hydroxybutyrate, milk urea nitrogen, and somatic cell count. The analyte may be only progesterone or β-hydroxybutyrate.

The method may further comprise permanently storing raw spectrograms and associating each DHIA-equivalent value with a model revision identifier to enable retroactive re-calculation. The method may further involve a system comprising an inline NIR milk sensor, an on-farm data concentrator, and a cloud processor configured to perform the method above. In this system, the milk sensor performs on-board crude milk detection using vector distance thresholding, designed to reduce transmitted data by at least 70%.

The regression model for progesterone may be configured to achieve R²≥0.92 and RMSEP≤2.5 ng/mL against reference Enzyme-Linked Immunosorbent Assay (ELISA) on blind data. The method may further include correlating the DHIA-equivalent value with an individual animal identifier. The method may further involve trimming configured to remove at least the first 5% and last 5% of samples. The method may further involve the transformation function configured to differ by analyte. The method may include the use of a single progesterone value for automated heat detection or pregnancy confirmation.

A non-transitory computer-readable medium may contain instructions that, when executed, cause a processor to perform any or all of the method steps disclosed herein. The DHIA-equivalent transformation may yield a mean deviation≤0.4% fat from certified DHIA laboratory results. The regression model may be selected from among partial least squares, polynomial regression, bicubic regression, or other appropriate models. The method may further comprise a cloud-based Mahalanobis-distance contamination filter on bubble-sensitive wavelengths. The method may further include storing raw spectrograms indefinitely and reprocessing them with future improved models. The transformation function may reconstruct the volume-weighted average produced by constant-volume drips into a DHIA proportional sampling jar.

In an alternative embodiment, the method for inline determination of progesterone concentration in milk (bovine and other animals) comprises acquiring a plurality of NIR spectrograms of flowing milk during a milking session, applying a regression model trained on a set of reference ELISA values to produce a progesterone time-series, transforming the time-series into a single DHIA-equivalent progesterone value using the transformation function as a weighted average Σ wᵢ c(tᵢ)/Σ wᵢ, the weights wᵢ empirically configured to minimize deviation from physical DHIA jar results, and outputting the value for reproductive management; wherein the method can be configured to achieve R²≥0.92 against laboratory ELISA, though results may vary.

Further disclosed is a real-time inline milk analysis system comprising: a plurality of NIR milk sensors, each configured to acquire at least one spectrogram every five to ten seconds while milk flows past the sensor; an on-farm data concentrator (hereinafter which can also be termed “conbox”) in communication with the NIR milk sensors, the concentrator configured to timestamp and buffer raw spectrograms; a cloud-based processing platform in network communication with the concentrator; and a non-transitory computer-readable medium containing instructions that configure the cloud-based platform to determine a DHIA-equivalent milk analyte concentration from inline near-infrared (NIR) measurements. The on-farm data concentrator or conbox is the physical box/computer installed in the milking parlor or equipment room that collects, timestamps, buffers, and forwards the raw spectrograms from the inline NIR sensors to the internet/cloud.

In some embodiments of the real-time inline milk analysis system, each NIR milk sensor includes an on-board microcontroller configured to execute a crude milk-detection filter using vector-distance thresholding and to transmit spectrograms.

In some embodiments of the real-time inline milk analysis system, the cloud-based platform includes a DHIA-equivalent transformation module configured to convert a trimmed time-series of analyte concentrations into a single value that replicates the volume-weighted average produced by constant-volume drip sampling into a physical DHIA proportional sampling jar.

In some embodiments of the real-time inline milk analysis system, the transformation module implements a clipped rectangular integration over the central 70-95% of the milking interval.

In some embodiments of the real-time inline milk analysis system, the transformation module implements a weighted average using weights empirically optimized on paired inline and DHIA-jar laboratory data.

Some embodiments of the real-time inline milk analysis system further comprise a cloud database configured to store every raw spectrogram indefinitely and to associate each DHIA-equivalent result with a model revision identifier.

In some embodiments of the real-time inline milk analysis system, the cloud-based platform includes a progesterone regression model trained to achieve R²≥0.92 and RMSEP≤2.5 ng/mL against reference ELISA values on blind data.

In some embodiments of the real-time inline milk analysis system, the progesterone regression model and the DHIA-equivalent transformation module together produce a single progesterone value per milking that deviates≤1.1 ng/mL from the ELISA result obtained on the corresponding physical DHIA jar sample.

Some embodiments of the real-time inline milk analysis system further comprise an animal identification interface configured to correlate each DHIA-equivalent result with an individual dairy animal identifier.

Some embodiments of the real-time inline milk analysis system further comprise a retroactive re-processing engine configured to apply a newer revision of a regression model or transformation function to the entire corpus of historically stored raw spectrograms without requiring new farm data collection.

A general object of the invention is to provide real-time milk analysis on-site without requiring data transfer off-site. The invention may still be used when data is transferred off-site, but users will have the flexibility to perform some or all of the analysis on-site.

A further object of the invention is to provide 2-4 data points per cow per day, compared to one per month, at no incremental cost per sample.

These and other objects, features, and advantages of the present invention will become readily apparent upon a review of the following detailed description of the invention, in view of the drawings and claims.

### BRIEF DESCRIPTION OF THE DRAWINGS

The nature and mode of the operation of the present invention will now be more fully described in the following detailed description of the invention, taken with the accompanying drawing figures, in which:

FIG. 1 illustrates a schematic of a system of performing an inline real time analysis of a milk sample within a dairy cow milking system.

FIG. 2 illustrates a flow chart of a method of performing an inline real time analysis of a milk sample while using a dairy cow milking system.

FIG. 3 is a table that illustrates reproducibility (R) limits for milk analysis (laboratory, at-line, and in-line recommendations) and percentage of NIR prediction residuals (RES) equal to or below R (RES≤R, %) known in the prior art.

FIG. 4A is a graph that illustrates mean-centered first Savitzky-Golay derivative of absorbance derived from transmittance spectra (400-2,450 nm) of 300 raw milk samples, with the most important absorption bands for fat and CP, known in the prior art.

FIG. 4B is a graph that illustrates milk fat content determination discussed in the prior art.

FIG. 4C is a graph that illustrates milk total protein content determination discussed in the prior art.

FIG. 4D is a graph that illustrates lactose content determination discussed in the prior art.

FIG. 4E1 is a graph that illustrates a Near-infrared (NIR) spectra (851 to 1,649 nm) of raw milk from an in-line measurement setup in diffuse reflectance mode, where typical spectra of milk during 1 cow milking (n=12).

FIG. 4E2 is a graph illustrating an NIR spectra of all raw milk samples (n=785).

FIG. 4E3 is a graph illustrating a normalized NIR reflectance spectra of all raw milk samples (n=785).

FIG. 5 illustrates a schematic of the disclosed real-time inline milk analysis system,

FIG. 6 illustrates a schematic of an inline NIR milk sensor,

FIG. 7 illustrates a schematic of on-sensor milk analysis, and

FIG. 8 illustrates a flow chart.

### DETAILED DESCRIPTION OF THE INVENTION

Following are detailed descriptions of various related concepts related to, and embodiments of, methods and apparatus according to the present disclosure. It should, however, be understood that this disclosure is not limited to the particular methodology, materials, and modifications described and, as such, may, of course, vary. It is also understood that the terminology used herein is for the purpose of describing particular aspects only and is not intended to limit the scope of the claims.

Furthermore, it should be appreciated that drawings are representative to illustrate the inventive concepts herein and may not be to scale. Also, like drawing numbers on different drawing views identify identical, or functionally similar, structural elements where there could appear some variations on exactness where exactness is not material to the inventive concept herein. It is to be understood that the claims are not limited to the disclosed aspects.

Unless defined otherwise, all technical and scientific terms used herein have the same meaning as commonly understood to one of ordinary skill in the art to which this disclosure pertains. It should be understood that any methods, devices, or materials similar or equivalent to those described herein can be used in the practice or testing of the example embodiments.

It should be appreciated that the term “substantially” is synonymous with terms such as “nearly,” “very nearly,” “about,” “approximately,” “around,” “bordering on,” “close to,” “essentially,” “in the neighborhood of,” “in the vicinity of,” etc., and such terms may be used interchangeably as appearing in the specification and claims. It should be appreciated that the term “proximate” is synonymous with terms such as “nearby,” “close,” “adjacent,” “neighboring,” “immediate,” “adjoining,” etc., and such terms may be used interchangeably as appearing in the specification and claims. It should be appreciated that the term “distal” and comparably related terms denoting further-away portions of an item are antonymous to proximal portions of the co-described item as those portions of items may be termed. The term “approximately” is intended to mean values within ten percent of the specified value.

It should be understood that the use of “or” in the present application is with respect to a “non-exclusive” arrangement unless stated otherwise. For example, when saying that “item x is A or B,” it is understood that this can mean one of the following: (1) item x is only one or the other of A and B; (2) item x is both A and B. Alternately stated, the word “or” is not used to define an “exclusive or” arrangement. For example, an “exclusive or” arrangement for the statement “item x is A or B” would require that x can be only one of A and B. Furthermore, as used herein, when referring to a set or group of items, for illustration (A, B, C) the term “at least one or more . . . and . . . ” such as in “at least one or more of A, B, and C” is intended to include any to all of the denoted set or group of items, i.e. it could include just one item from the set or group, it could include all of the items from the set or group, and it could include any other combination of the set or group of items that is greater than one item and less than all of the items, the illustrated example having three items meaning there are up to seven non-ordered combinations A, B, C, AB, AC, BC, ABC. Other numbers of items would have maximum combination possibilities calculated accordingly.

Moreover, as used herein, the phrases “comprises at least one of” and “comprising at least one of” in combination with a system or element is intended to mean that the system or element includes one or more of the elements listed after the phrase. For example, a device comprising at least one of: a first element; a second element; and, a third element, is intended to be construed as any one of the following structural arrangements: a device comprising a first element; a device comprising a second element; a device comprising a third element; a device comprising a first element and a second element; a device comprising a first element and a third element; a device comprising a first element, a second element and a third element; or, a device comprising a second element and a third element. A similar interpretation is intended when the phrase “used in at least one of:” is used herein.

The term between as used in this disclosure includes the value denoting the endpoints of the set. For illustration, a value between A and B includes A and any value A that is less than B and it includes B and any value that is greater than A, according to the discrete or continuous limitations of what value in the set can constitute a value.

While inventive concepts have been described above in terms of specific embodiments, it is to be understood that the inventive concepts are not limited to these disclosed embodiments. Upon reading the teachings of this disclosure, many modifications and other embodiments of the inventive concepts will come to mind of those skilled in the art to which these inventive concepts pertain, and which are intended to be and are covered by both this disclosure and the appended claims. It is indeed intended that the scope of the inventive concepts should be determined by proper interpretation and construction of the appended claims and their legal equivalents, as understood by those of skill in the art relying upon the disclosure in this specification and the attached drawings.

Embodiments of the invention relate to a real-time, inline milk analysis system that produces DHIA-equivalent component values along with progesterone and p-hydroxybutyrate concentrations, taken directly from flowing bovine milk using near infrared (NIR) spectroscopy.

Primary embodiments involve obtaining an optimized weighted average. The DHIA-equivalent analyte concentration for a single milking session can be calculated as:

$${\overline{c}}_{DHIA} = \frac{{\sum }_{i = {i}_{start}}^{{i}_{end}} {w}_{i} c ( {t}_{i} )}{{\sum }_{i = {i}_{start}}^{{i}_{end}} {w}_{i}}$$

where
\tilde{c}_{DHIA} is the single DHIA-equivalent analyte concentration reported to the farmer and used for official payment, herd management, or reproductive decisions;
c(tᵢ) is the predicted analyte concentration (e.g., fat (%), progesterone (ng/mL), BHB (mmol/L), etc.) from the validated NIR spectrogram acquired at time index i;
iₛₜₐᵣₜ and i_{end} are the first and last indices of the trimmed milking interval (after automatic removal of pre-milk, post-milk, and contaminated portions);
wᵢ≥0 are non-negative weighting coefficients that are empirically optimized on a training dataset (high-frequency inline predictions vs. certified laboratory results obtained from the corresponding physical DHIA proportional sampling jar) so as to minimize the objective function.

$${\sum }_{k = 1}^{{N}_{train}} \vert {\overline{c}}_{DHIA}^{( k )} ( w ) - {c}_{lab}^{( k )} \vert$$

In the most robust implementations, the weights wᵢ are determined separately for each analyte (fat, protein, progesterone, β-hydroxybutyrate, etc.) because milk component secretion profiles differ across the milking session.

This weighted average exactly reconstructs the volume-weighted mean that would be produced by the constant-volume drip mechanism of a physical DHIA proportional sampling jar, thereby achieving DHIA-equivalence to within the tolerances stated elsewhere in this specification (e.g., mean fat deviation≤0.04%, progesterone deviation≤1.1 ng/mL).

Preferred embodiments may further include clipped rectangular (mid-milking) averages. Representatively, the DHIA-equivalent analyte concentration may be computed as the unweighted arithmetic mean over only the central high-flow portion of the milking session:

$${\overline{c}}_{DHIA} = \frac{1}{{N}_{high}} {\sum }_{i ∈ {𝒥}_{high}} c ( {t}_{i} )$$

where
N_{high} is the number of valid measurements retained in the central high-flow subset;
J_{high} is the contiguous subset of time indices defined as follows:
   where the full trimmed milking interval (after removal of pre-milk, post-milk, and contaminated spectra) includes Nₜᵣᵢₘ consecutive valid measurements indexed i=1, 2, . . . , Nₜᵣᵢₘ in chronological order. Then

$${𝒥}_{high} = \{ i \vert ⌊ {α}_{low} \cdot {N}_{trim} ⌋ \leq i \leq ⌈ {α}_{high} \cdot {N}_{trim} ⌉ \}$$

with the exclusion percentages set such that the retained central portion falls between 70% and 95% of the trimmed milking duration. Representative values are α_{low}∈[0.025,0.15] and α_{high}∈[0.85,0.975], with the preferred default being α_{low}=0.10 and α_{high}=0.90 (i.e., discarding the first and last 10%).

This embodiment requires no training data for the transformation itself, and it nevertheless achieves DHIA-equivalence well within official laboratory tolerances (e.g., mean absolute fat deviation≤0.05%, progesterone 1σ deviation≤1.2 ng/mL on blind validation sets of >8,000 milking sessions).

Preferred embodiments further may include trapezoidal Volume-Weighted Integration, which may be represented as follows. The DHIA-equivalent analyte concentration is computed via trapezoidal numerical integration over the trimmed milking interval:

$${\overline{c}}_{DHIA} = \frac{1}{{T}_{trim}} {\sum }_{i = {i}_{start}}^{{i}_{end} - 1} \vert \frac{c ( {t}_{i} ) + c ( {t}_{i + 1} )}{2} \cdot ( {t}_{i + 1} - {t}_{i} )$$

where
\tilde{c}_{DHIA} is the single DHIA-equivalent analyte concentration;
tᵢ are the actual timestamps (in seconds) of the validated NIR spectrograms within the trimmed milking interval;
iₛₜₐᵣₜ and i_{end} are the first and last indices of the trimmed, contiguous, valid time-series (after removal of pre-milk, post-milk, and bubble-contaminated spectra);
Tₜᵣᵢₘ=tᵢ_{_{end}}−tᵢ_{ₛₜₐᵣₜ} is the total duration (in seconds) of the trimmed milking interval.

Because measurements are acquired at high frequency (≥1 spectrogram every 5 to 10 seconds, typically every 1-3 seconds), the trapezoidal integration provides an approximation to the true volume-weighted average even when the instantaneous flow rate varies significantly during the milking session.

The various embodiments of the invention provide for a system and a method to conduct for inline estimation of milk parameters such as fat, protein, lactose, somatic cell contents (SCC), and progesterone that can be performed in real time, using an NIR spectroscopy based system, during the milking process exhibiting commercially acceptable levels of accuracy. Generally, the NIR spectrum lies between 700 to 2500 nanometers (nm). This broad range can be divided into two segments of NIR wavelengths: the shorter wavelengths segment from 700-1100 nm, and the longer wavelengths between 1100-2500 nm. However, for practical and cost-effective implementations, it may not necessary to include the entire range from 700-2500 nm in the milk composition estimation, since there appears to be no measurable improvement in accuracy when the two segments are combined. The NIR spectrum of longer wavelengths between 1000-2500 nm appears to give the best results, which are close to the standard laboratory method. Conversely, longer NIR wavelengths do appear to have two disadvantages: optical sensors needed for them are expensive; and the optimal path length of the light is 0.5 to 1 mm. The small width, makes it unsuitable for inline measurements and a bypass is necessary. When used for online measurements, care must be taken that a collection or analysis tube or vessel used is narrow.

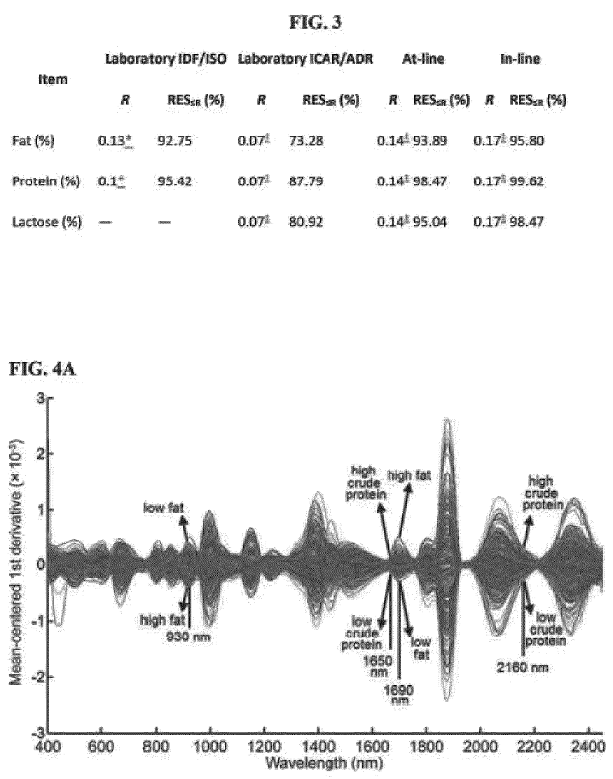

In contrast, shorter wavelengths, use a light pathway between 10 to 13.5 mm, so the online/inline bypass tube can be wider than when longer wavelengths are used. The optical sensors for the segment of shorter NIR wavelengths are inexpensive. The at-line measurements by shorter wavelengths (700 to 1100 nm) are comparable in accuracy to the 1100-2500 nm range. The online estimations by the shorter wavelengths are satisfactory since it appears that the lower accuracy by shorter wavelengths, during online and inline estimations, is commercially acceptable and is to be expected. Even various standards set by international and national agencies, for milk quality, like the ICAR or ISO accept lower accuracy for inline estimations in comparison to laboratory procedures. See FIG. 3 (Table 1), where the error (Res_{R}) for inline can be higher than laboratory or at-line results. In particular, FIG. 3 illustrate reproducibility (R) limits for milk analysis (laboratory, at-line, and in-line recommendations) and percentage of NIR prediction residuals (RES) equal to or below R (RES≤R, %). Symbols indicate the reference(s) corresponding to the value of R: an asterisk (*) indicates adapted according to IDF standard 141C:2000 (IDF, 2000) and ISO standard 9622. (ISO, 1999; SD of fat: 0.045%; SD of protein: 0.035%); a plus sign (+) indicates ADR recommendation 1:13 (ADR, 2002); a dagger (‡) indicates the ICAR standard (ICAR, 2010); a bullet (⋅) indicates adapted according to the ICAR (2010) and ADR (2002) recommendations,” Aernouts et al 2011. (Credits: DOI:https://doi.org/10.3168/jds.2011-4354)

Online measurements can monitor individual cows daily, which would give more information to help decision-making by dairy owners, than a perfect estimation done once in two to three weeks. The shorter NIR wavelengths are suitable for spectroscopy estimations as they will allow for rapid and non-destructive milk composition estimation in real-time and improve the quality and quantity of dairy milk production.

The mode of NIR sensor measurement commonly reported for milk is diffuse transmittance. Data is collected as transmittance spectra and recorded in the linked computer as absorbance [i.e., log (1/T)]. In some cases, reflectance can give better results for fat and protein measurements in the 1100-2500 nm range, however, lactose is more challenging for estimating by reflectance accurately. So, the transmittance mode is better than reflectance to find milk composition. The suitable wavelengths chosen are those that showed the most variations for each parameter. In other words, these wavelengths are sensitive to changes in concentrations of the respective parameter. For the NIR region from 700 to 1100 nm, where inexpensive online sensors could be used, the highest positive coefficients for measuring the various parameters are given.

Fat Estimation—Mode: Data is collected as transmittance spectra at 2-nm intervals and recorded in the linked computer as absorbance [i.e., log (1/T)]. Spectral data collection can have a path length up to 10 mm. Best Wavelengths: To estimate fat, the best wavelengths to use are 730, 770, 930, 968, 990, 1026, 1076, and 1092 nm; 930 nm was the best.

Lactose—Mode: Data is collected as transmittance spectra at 2-nm intervals and recorded in the linked computer as absorbance [i.e., log (1/T)]. Spectral data collection can have a path length up to 10 mm. Best Wavelength: For lactose, the best wavelengths are 734, 750, 786, 812, 908, 974, 982, and 1064 nm; and 1064 nm were the best.

Protein-Mode: Data is collected as transmittance spectra at 2-nm intervals and recorded in the linked computer as absorbance [i.e., log (1/T)]. Spectral data collection can have a path length up to 10 mm. Best Wavelength: To estimate proteins use 726, 736, 760, 776, 880, 902, 952, and 1034 nm.

Progesterone—There is only one study, so far, to estimate progesterone in milk using NIR spectroscopy by Iweka et al 2020. This data is for inline estimation. Mode: Use absorbance data between 700-1050 nm at 1-nm intervals. Wavelengths: The two most important wavelengths are 740 and 840 nm. But these wavelengths are also said to be relevant for fat, proteins, and lactose. No further details are available for any other parameters by these scientists, except that they used the spectrum between 700-1100 nm.

SCC (somatic cell count)—Two studies successfully estimated SCC using the short-wavelength segment, but do not mention the specific NIR wavelengths they found useful. A third study was found that makes recommendations in the shorter wavelength range. The results obtained were within the range needed for daily real-time monitoring of milk for mastitis detection. Mode: Use transmittance data between 700-1100 nm at 2-nm intervals. The sample size used was 10 ml of milk in a test tube with 12 mm inner diameter and 16 mm outer diameter. So the light length that can be used is 16 mm. Wavelengths: The best positive correlations are 832, 926, and 960 nm. The highest negative correlation was at 1004 nm. Other important wavelengths are 762, 780, and 870 nm. The results reflect the changes in protein and fat that occurs due to changes in health of the cows.

Recommendations for Longer NIR Wavelengths Segment—Since the region of longer NIR wavelengths (1100 to 2400 nm), gives more precise results, these findings are also mentioned here, in case it is chosen for use. Results from two studies are covered below.

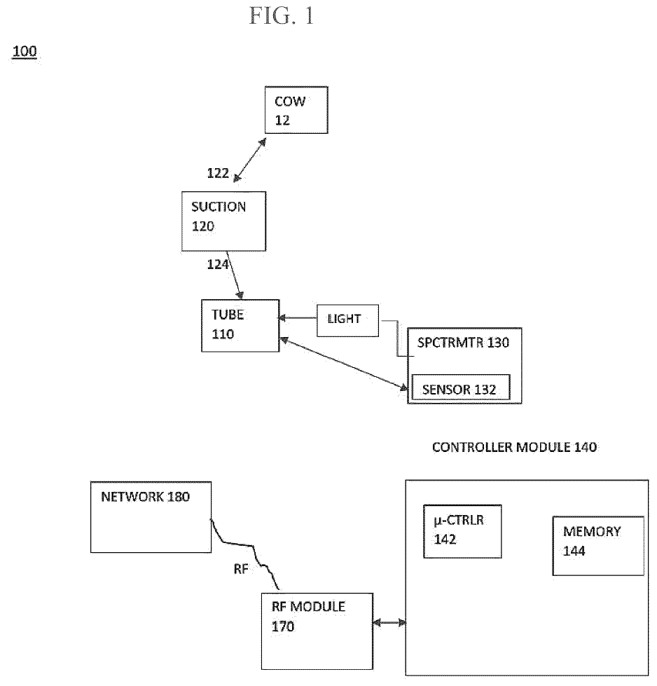

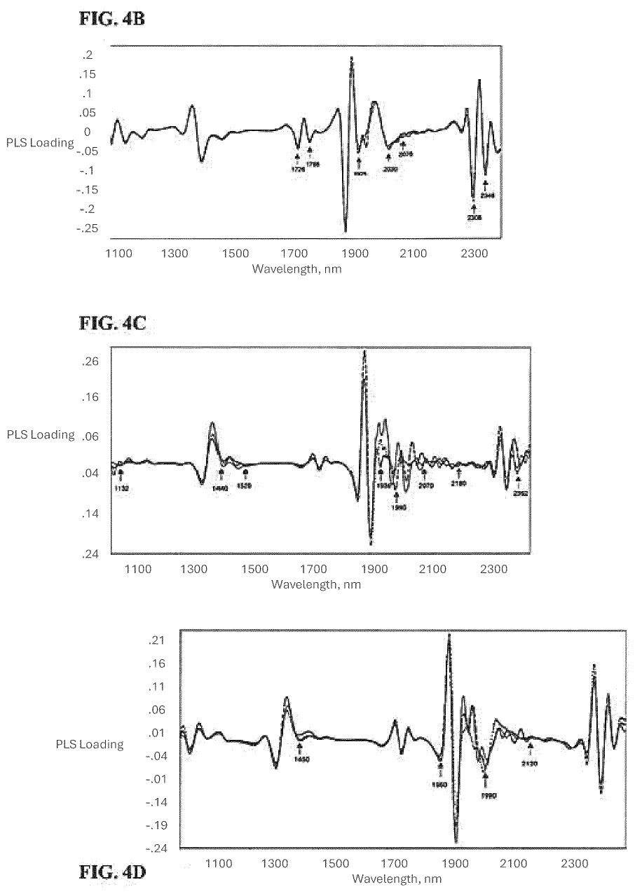

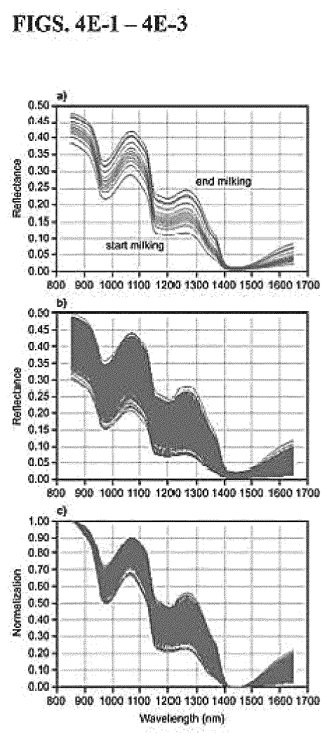

Fat Estimation—Mode: Data is collected as transmittance spectra at 2-nm intervals and recorded in the linked computer as absorbance [i.e., log (1/T)], in both studies (see FIG. 4A). FIG. 4A (FIG. 1 in the article) is a graph that illustrates mean-centered first Savitzky-Golay derivative of absorbance derived from transmittance spectra (400-2,450 nm) of 300 raw milk samples, with the most important absorption bands for fat and CP. Aernouts et al. (2011). (Image credits: DOI:https://doi.org/10.3168/jds.2011-4354)

Wavelengths: Based on two studies, the best wavelength suggested here is 930 nm. The study that researched online estimations also recommends 1690 nm, see FIG. 1 above.

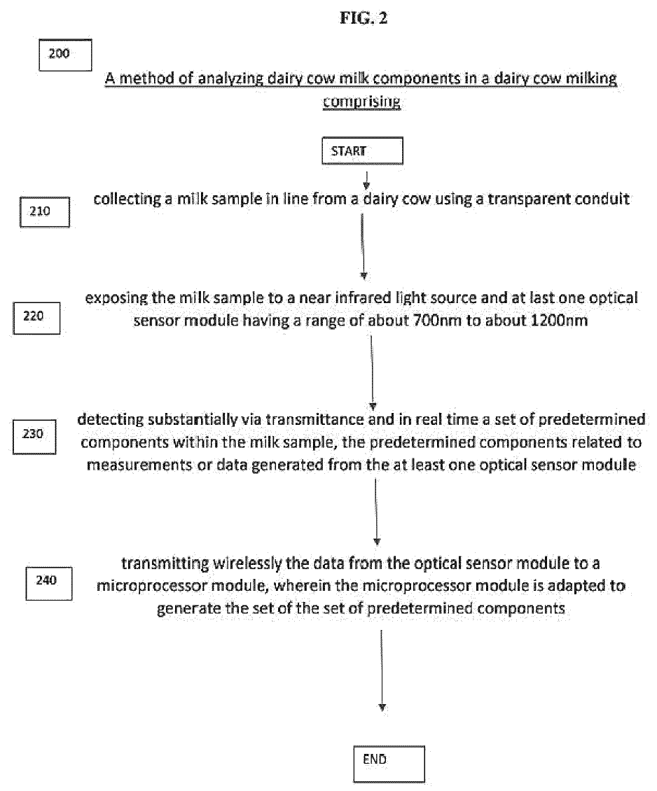

The other study based on at-line estimation recommends 930, 1726, 1760, 2308, 2348, 1160, 1210, 2354 nm; see FIG. 4B (FIG. 2 in the article) is a graph that illustrates milk fat content determination, (Tsenkova et al, 2000). (Image credits: https://doi.org/10.2527/2000.783515x)

Protein Estimation—Mode: Data is collected as transmittance spectra at 2-nm intervals and recorded in the linked computer as absorbance [i.e., log (1/T)]. Wavelengths: Based on the online study the wavelengths recommended are 1650 and 2160 nm, see FIG. 4A (FIG. 1 in the article).

Based on the at-line study the useful wavelengths are 1132, 1460, 1490, 1520, 1990, 2030, and 2070 nm. Also, bands from 1,460 to 1,520 nm, 1,980 to 2,070 nm, and 2,170 to 2,180 nm, respectively, were absorbed by proteins, see FIG. 4C (FIG. 3 in the article) which illustrates the milk total protein content determination. Milk total protein content determination, (Tsenkova et al., 2000). (Image credits: https://doi.org/10.2527/2000.783515x)

Lactose—Mode: Data is collected as transmittance spectra at 2-nm intervals and recorded in the linked computer as absorbance [i.e., log (1/T)]. Wavelengths: Based on the online study the most wavelength recommended is 1490, and also the band between 1480-1500 nm. According to the at-line study, the important wavelengths are 1406 nm, 2011 nm, 1860 nm, and the regions 1438 to 1450 nm, 1460 to 1500 nm, and from 1920 to 2120 nm; see FIG. 4D (see FIG. 4 in the article) illustrating Lactose content determination, (Tsenkova et al, 2000). (Image credits: https://doi.org/10.2527/2000.783515x)

Somatic Cell Count (SCC)—There is only one study that gives details of the wavelengths that is useful for SCC estimation. NIR determination of log SCC was based on relative changes in milk composition affecting milk spectral changes—lactose and proteins. Cows suffering from mastitis produce less lactose, and the type of protein produced changes during illness. Mode: NIR transflectance (T) spectra were collected in a flow cell with a path length of 0.2 mm expressed as absorbance—log(1/T). Wavelength: Real-time analysis is possible by using wavelengths 1412, 1886, 1920, 1996, 2020, 2186, 2298, and 2498 nm.

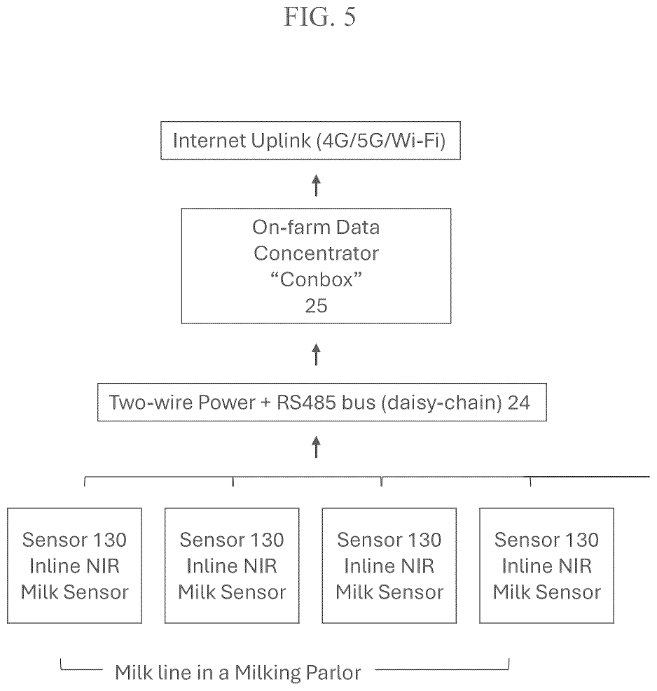

There are a few points that must be kept in mind during inline NIR spectroscopy estimation of milk composition during milking. These are: Milking Dynamics: Expect a shift in spectra from beginning to the end of milking, see FIG. 4E-1 (FIG. 5a in the article). This could be because of a change in the composition of milk, and also due to the mixture of air with milk that scatters light. In the article by Melfsen, FIGS. 4E-E-3 (FIGS. 5a-5c) Near-infrared (NIR) spectra (851 to 1,649 nm) of raw milk from an in-line measurement setup in diffuse reflectance mode. FIG. 4E-2 (FIG. 5a) illustrates typical spectra of milk during 1 cow milking (n=12). FIG. 4E-2 illustrates (FIG. 5b in the article) spectra of all raw milk samples (n=785), while FIG. 4E-3 (FIG. 5c in the article) illustrates normalized NIR reflectance spectra of all raw milk samples (n=785),” (Melfsen et al. 2012). (https://doi.org/10.3168/jds.2012-5388)

Interference by Water: Very low reflectance, absorbance, and transmittance values were recorded for estimation of all parameters, around 960, 970, 1190, 1450, and 1950 nm and above 2400 nm, due to the high absorbance of these wavelengths by water in the milk.

Chemometrics: Water interaction with NIR masks any other interactions. Hence, raw spectra cannot be used to estimate the parameters. The spectral data will have to be pretreated (smoothing and derivative transformation) before using in a chemometrics model, See FIGS. 4E-1 through 4E-3 (FIGS. 5b and c in the Melfsen article).

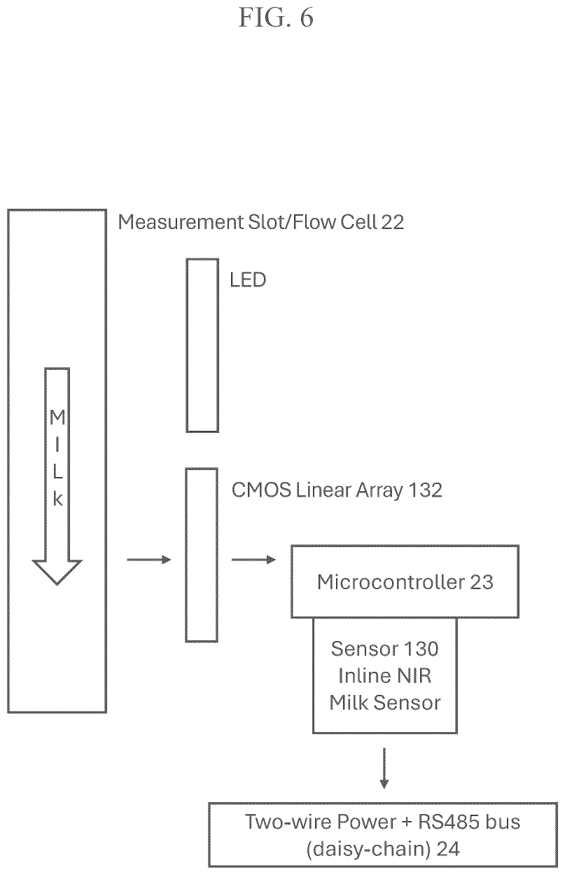

Added, and referring now to FIGS. 5 and 6, FIG. 5 illustrates a schematic of a system 100 of performing an inline real time analysis of a milk sample 14 from a dairy cow 12 within a dairy cow milking system 10. System 100 includes a milk collection vessel or tube or conduit 110 (preferably transparent or translucent) and includes a suction apparatus 120 having an inlet 122 and an outlet 124, the outlet 124 coupled to the milk collection vessel 110 and the inlet 122 designed to be coupled to dairy cow 12. System 100 further includes a near infrared (NIR) sensor 130 (a spectrometer that includes CMOS sensor 132) designed to provide light and collect light, in a range of about 700 nm to about 1200 nm, to the milk collection vessel 110. Light is collected primarily, but not necessarily limited to, transmittance or absorbance of the near infrared light. System 100 also includes a controller module 140 including a microcontroller 142 and a memory module 144, the controller module 140 designed to receive data from the NIR sensor 130, a set of data indicative of data measurements 160 of a set of predetermined milk components.

In another embodiment, system 100 includes a radio frequency (RF) wireless communications module 170 designed to transmit data of at least one of the set of predetermined milk components including fat, protein, lactose, somatic cell contents (SCC), and progesterone to an internal or external network 180 for further processing of data.

Referring now to FIG. 2, there is illustrated a flowchart of a method 200 of collecting a milk sample 14 in line from a dairy cow 12 while on a dairy cow milking system 10. Method 200 includes step 210 of exposing the milk sample 14 to a NIR sensor 130 having near infrared light source and an optical sensor. The light source having a range of about 700 nm to about 1200 nm. Step 220 includes detecting in real time a set of predetermined components within the milk sample, such that the predetermined components are detected with a dairy cow milking system 10. In this example embodiment, the set of predetermined milk components includes one or more of protein, fat, progesterone, and somatic cell count. In method 200 the milk components indicate bovine conditions that affect milk production including one or more of mastitis, estrus, dehydration and starvation.

Added is the following. FIG. 5 presents an overview of the real-time, inline milk analysis system. A plurality of inline NIR milk sensors 130 are installed in the milk lines of a milking parlor. Each NIR milk sensor 130 is connected via a bus, such as a two-wire power+RS485 bus 24, to an on-farm data concentrator 25 (the representative example referred to as the “conbox”). Farm data concentrator/conbox 25 forwards raw spectrograms through an internet uplink to a cloud-based processing platform.

Representative conbox 25 serves as a data aggregation and communication hub, connecting multiple milk sensors to the cloud. Key features include:
1. Data Collection: Supports up to four sensor strings, each handling up to 64 sensors, using RS485 communication. Initially designed for polled data collection, future iterations may implement carrier-sense multiple access for autonomous sensor transmission.
2. Internet Connectivity: Connects to the internet via wired Ethernet, cellular, Wi-Fi, or satellite, timestamping sensor data and forwarding it to the cloud using MQTT over an encrypted TLS connection to AWS IoT Core.
3. Power Management: Currently uses four separate power supplies for fault isolation, with plans for a single power supply with monitored outputs to enable automatic short-circuit protection and remote resets.
4. Firmware and Configuration Updates: Facilitates remote firmware and configuration updates to sensors, ensuring system reliability and adaptability.

NIR milk sensor 130 is the core component for data acquisition, utilizing NIR spectroscopy to analyze milk composition. Key features include, representatively:
1. NIR Spectrometer: Employs a diffraction grating-type spectrometer with a CMOS image sensor 132 tuned for NIR wavelengths. The spectrometer uses a constant-current LED light source to ensure consistent brightness across units and over time, compensating for temperature variations. Light is focused through lenses, dispersed into a rainbow effect, and captured by CMOS sensor 132, where each pixel corresponds to a specific wavelength (1 nm).
2. Data Acquisition: A digital clock drives an analog multiplexer to output pixel voltages proportional to light intensity. A 16-bit analog-to-digital converter digitizes these voltages at up to 10 million samples per second, capturing data from 256 pixels.
3. Measurement Tube: A food-grade, high-density polyethylene tube (approximately 1-inch cross-section) narrows to a slot for efficient milk measurement, requiring only partial filling. Iterations may incorporate a single-piece, injection-molded design with opaque hose fittings to prevent external light interference and eliminate gaskets, reducing maintenance and leakage risks.
4. Power and Communication: Operates on a wide input voltage range (9-48 V) to minimize wiring diameter. An innovative two-wire system combines DC power and RS485 communication using Manchester encoding, with a bridge rectifier to ensure polarity insensitivity. A planned logic inverter may allow the microcontroller to adapt to reversed communication lines, simplifying installation.

FIG. 6 is a schematic of the inline NIR milk sensor 130. Milk flows through flow cell 2. A constant-current LED illuminates the milk, and transmitted light is diffracted and imaged onto a Complementary Metal-Oxide-Semiconductor (CMOS) linear array 132, also termed as CMOS sensor 132, such as a 256-pixel CMOS linear array. Microcontroller 23, on sensor, executes firmware that performs crude milk detection and transmits milk spectrograms over bus 24.

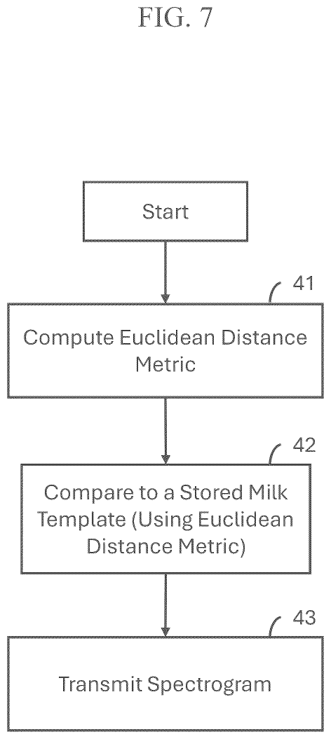

FIG. 7 is a flowchart of on-sensor milk analysis executed by the microcontroller 3. Each newly acquired spectrogram vector is compared to a stored milk template using a Euclidean distance threshold. Only spectrograms classified as milk are transmitted, reducing uplink bandwidth by 70-90%. Steps include 41, compute Euclidean distance metric, 42, compare to a stored milk template (using Euclidean distance metric), and 43, transmit spectrogram.

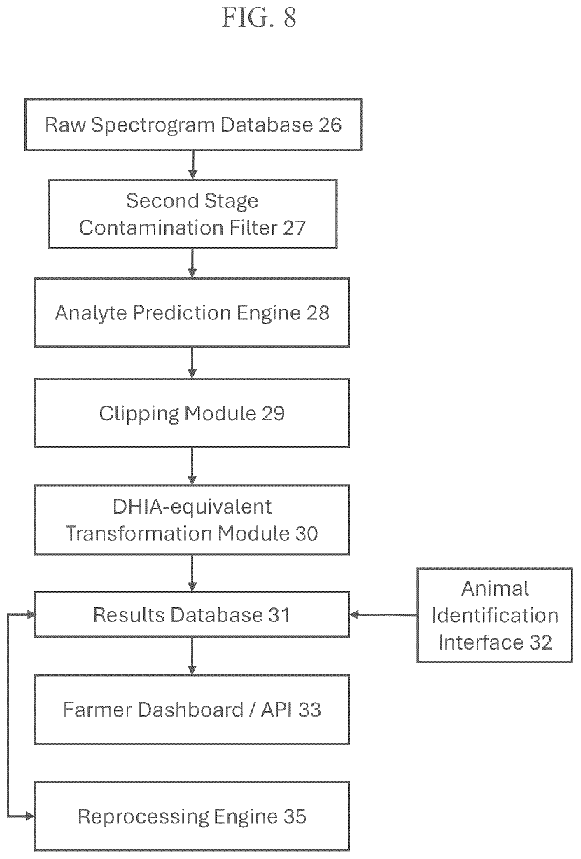

FIG. 8 illustrates a representative complete cloud data flow schematic. Raw spectrograms are ingested into an immutable cloud raw spectrogram database 26. A second-stage contamination filter 27 applies a Mahalanobis-distance outlier test on bubble-sensitive wavelengths and flags suspect records. Validated spectrograms are then processed by the Cyclops analyte prediction engine 28, which applies versioned regression models (PLS, polynomial, or bicubic) to produce a high-frequency time series of analyte concentrations. The milking interval detection and clipping module 29 automatically identifies the longest continuous sequence of valid spectrograms and removes the first 5-15% and last 5-15% of samples to exclude pre-milk residuals and post-milking rinse.

The cloud platform processes raw sensor data to deliver actionable insights. Key components include:
1. Data Ingestion and Storage: Sensor data, formatted, representatively, as 256-pixel intensity arrays with sensor unique IDs (UIDs) and timestamps, is stored indefinitely in a database. Calibration factors from the spectrometer manufacturer are applied to account for mechanical alignment variations.
2. Cyclops Analysis Engine 28: Processes calibrated data to quantify analytes (e.g., protein, fat, lactose, progesterone, mycotoxins) using machine learning models. Outputs are stored in a results database with metadata, including sensor UID, cow ID, and model version.
3. Cow Identification: Animal identification interface 32 correlates milk quality data with cow IDs using external herd management software (e.g., DairyPlan, NEAP RFID systems) or proposed location-based systems (e.g., GPS or LoRa-based tags), improving accuracy over traditional RFID methods.
4. Data Processing and Filtering: Averages data to replicate standard Dairy Herd Improvement Association (DHIA) test results, filtering out anomalies due to the bursty nature of milk flow. Scalable architecture leverages AWS's parallel processing capabilities to handle high data volumes.

FIG. 8 further illustrates that DHIA-equivalent transformation module 30 receives the trimmed time-series and converts it into a single value that mathematically replicates the volume-weighted average produced by constant-volume drip sampling into a physical DHIA proportional sampling jar. Three embodiments are disclosed: (i) clipped rectangular integration over the central high-flow portion, (ii) an optimized weighted average with weights trained on thousands of paired inline/DHIA-jar datasets, and (iii) trapezoidal numerical integration across the trimmed interval. Results—including the single DHIA-equivalent value, cow identifier, timestamp, and the exact revision numbers of the regression model and transformation function used—are stored in results database 31.

Animal identification interface 32 correlates each milking session with an individual cow using either existing parlor RFID systems or a proprietary location-based identification system. Farmer dashboard/API 33 provides real-time access to DHIA-equivalent component values, progesterone-based heat alerts, and longitudinal trends.

Because raw spectrogram is stored indefinitely in database 26, retroactive re-processing engine 35 is designed to apply alternate or improved regression models or transformation functions to the entire historical corpus at any time, enabling continuous accuracy improvement and regulatory re-certification without new farm samples.

After application of the DHIA-equivalent transformation module 30, the single reported progesterone value is designed to deviate, representatively, by only 0.34±1.1 ng/mL from the certified laboratory ELISA performed on the corresponding physical jar sample.

While particular embodiments have been shown and described, persons of ordinary skill will recognize that numerous modifications (different wavelength counts, alternative regression techniques, additional analytes, hybrid transformation functions, etc.) may be made without departing from the scope of the claims. Where specific systems are referred to by a manufacturer's name or category name, equivalent systems may be substituted.

### LIST OF REFERENCE NUMERALS

1. cow milking
14. milk sample
12. dairy cow
10. dairy cow milking system
22. Measurement slot/flow cell
23. On-sensor microcontroller
24. Two-wire power+RS485 bus
25. On-farm data concentrator (“Conbox”)
26. Cloud raw spectrogram database
27. Second-stage contamination filter
28. Cyclops analyte prediction engine
29. Milking interval detection & clipping module
30. DHIA-equivalent transformation module
31. Results database (per-cow DHIA-equivalent values)
32. Animal identification interface
33. Farmer dashboard/API
35. Reprocessing Engine
100. System of performing inline real time
110. Milk collection vessel (jar) or tube or conduit (preferably transparent or translucent) and includes
120. Suction apparatus
122. Inlet 122
124. Outlet
130. Near infrared (NIR) spectrometer (which includes a CMOS sensor)
132. CMOS sensor (also termed CMOS array)
140. Controller module
142. Microcontroller 142
144. Memory module
140. Controller module
180. Internal or external network for further processing of data.

1. A method for determining a DHIA-equivalent milk analyte concentration from inline near-infrared (NIR) measurements taken during a single milking session of a dairy animal, comprising:
receiving a time-series of validated NIR spectrograms acquired at least every five seconds;
applying a regression model to generate a time-series of predicted analyte concentrations c(tᵢ);
automatically detecting and trimming pre-milk and post-milk portions;
mathematically transforming the trimmed time-series into a single DHIA-equivalent value using a transformation function configured to replicate constant-volume drip sampling of a DHIA jar; and
outputting said single DHIA-equivalent value.

2. The method of claim 1, wherein the transformation function is a clipped rectangular integration over the central 70-95% of the milking interval.

3. The method of claim 1, wherein the transformation function is a weighted average Σ wᵢ c(tᵢ)/Σ wᵢ, the weights wᵢ empirically configured to minimize deviation from physical DHIA jar results.

4. The method of claim 1, wherein the analyte is selected from fat, protein, lactose, progesterone, β-hydroxybutyrate, milk urea nitrogen, and somatic cell count.

5. The method of claim 4, wherein the analyte is progesterone or β-hydroxybutyrate.

6. The method of claim 1, further comprising permanently storing raw spectrograms and associating each DHIA-equivalent value with a model revision identifier to enable retroactive re-calculation.

7. The method of claim 1, wherein the regression model for progesterone achieves R²≥0.92 and RMSEP≤2.5 ng/mL against reference ELISA on blind data.

8. The method of claim 1, further comprising correlating the DHIA-equivalent value with an individual animal identifier.

9. The method of claim 1, wherein trimming removes at least the first 5% and last 5% of valid samples.

10. The method of claim 1, wherein the transformation function differs by analyte.

11. The method of claim 1, wherein the DHIA-equivalent transformation yields a mean deviation≤0.4% fat from certified DHIA laboratory results.

12. The method of claim 1, wherein the regression model is selected from partial least squares, polynomial regression, or bicubic regression.

13. The method of claim 1, further comprising a cloud-based Mahalanobis-distance contamination filter on bubble-sensitive wavelengths.

14. The method of claim 1, wherein raw spectrograms are stored indefinitely and may be re-processed.

15. The method of claim 1, wherein the transformation function reconstructs the volume-weighted average produced by constant-volume drips into a DHIA proportional sampling jar.

16. A method for inline determination of progesterone concentration in bovine milk, comprising:
acquiring a plurality of NIR spectrograms of flowing milk during a milking session, applying a regression model trained on a set of reference ELISA values to produce a progesterone time-series, transforming said time-series into a single DHIA-equivalent progesterone value using the transformation function of claim 3, and
outputting said value for reproductive management; wherein
the method achieves R²≥0.92 against laboratory ELISA.

17. A real-time, inline milk analysis system comprising:
a plurality of NIR milk sensors, each NIR milk sensor configured to acquire at least one spectrogram every ten seconds while milk flows past each of said NIR milk sensor;
an on-farm data concentrator in communication with the NIR milk sensors, the concentrator configured to timestamp and buffer raw spectrograms;
a cloud-based processing platform in network communication with the concentrator; and
a non-transitory computer-readable medium containing instructions that configure the cloud-based platform to determine a DHIA-equivalent milk analyte concentration from inline near-infrared (NIR) measurements.

18. The system of claim 17, wherein each NIR milk sensor includes an on-board microcontroller configured to execute a crude milk-detection filter using vector-distance thresholding.

19. The system of claim 17, wherein the cloud-based platform includes a DHIA-equivalent transformation module configured to convert a trimmed time-series of analyte concentrations into a single value that replicates the volume-weighted average produced by constant-volume drip sampling into a physical DHIA proportional sampling jar.

20. The system of claim 19, wherein the DHIA-equivalent transformation module implements a clipped rectangular integration over the central 70-95% of the milking interval.

21. The system of claim 19, wherein the DHIA-equivalent transformation module implements a weighted average using weights empirically optimized on paired inline and DHIA-jar laboratory data.

22. The system of claim 17, further comprising a cloud database configured to store raw spectrograms indefinitely and to associate each DHIA-equivalent result with a model revision identifier.

23. The system of claim 17, wherein the cloud-based platform includes a progesterone regression model trained to achieve R²≥0.92 and RMSEP≤2.5 ng/mL against reference ELISA values on blind data.

24. The system of claim 23, wherein the progesterone regression model and the DHIA-equivalent transformation module together produce a single progesterone value per milking that deviates≤1.1 ng/mL from the ELISA result obtained on the corresponding physical DHIA jar sample.

25. The system of claim 17, further comprising an animal identification interface configured to correlate each DHIA-equivalent result with an individual dairy animal identifier.

26. The system of claim 17, further comprising a retroactive re-processing engine configured to apply a newer revision of a regression model or transformation function to stored raw spectrograms without requiring new farm data collection.
